(19) United States
(12) Patent Application Publication
(10) Pub. No.: US 2013/0096379 A1
(43) Pub. Date: Apr. 18, 2013

(54) DOUBLE-LUMEN ENDOTRACHEAL TUBE DEVICES, SYSTEMS AND METHODS

(71) Applicant: Gary Stuart Goldberg, Phoenix, AZ (US)

(72) Inventor: Gary Stuart Goldberg, Phoenix, AZ (US)

(73) Assignee: Gary Stuart Goldberg, Phoenix, AZ (US)

(21) Appl. No.: 13/533,852

(22) Filed: Jun. 26, 2012

Related U.S. Application Data
(60) Provisional application No. 61/547,596, filed on Oct. 14, 2011.

(51) Int. Cl. A61M 16/04; A61B 1/06; A61B 1/267; A61B 1/04

(52) U.S. Cl. 600/109; 128/207.14; 128/207.15

(57) ABSTRACT

Embodiments of the present disclosure relate to a double-lumen endotracheal tube having an access sheath or optical system positioned between the two lumens. Embodiments of the the present disclosure are directed toward systems, devices, and methods that provide continuous visual input to a clinician during initial lung separation with the use of a double-lumen endotracheal tube device and throughout a treatment procedure to facilitate the ongoing maintenance of a proper position of the double-lumen endotracheal tube.

### CROSS-REFERENCE TO RELATED APPLICATION

This application is a non-provisional of and claims priority to U.S. Provisional No. 61/547,596, filed on Oct. 14, 2011 and entitled “Double-Lumen Endotracheal Tube”, wherein such provisional application is hereby incorporated by reference in its entirety.

### FIELD OF DISCLOSURE

The present disclosure relates to airway management devices, systems, and methods. More particularly, the present disclosure relates to devices, systems, and methods for endotracheal tubes.

### BACKGROUND OF THE DISCLOSURE

During a thoracic surgery, a clinician (e.g., an anesthesiologist or surgeon) is often required to “separate,” i.e., selectively ventilate, the lungs. This is usually done to facilitate the surgery by providing a deflated, motionless lung for the surgeon to operate on, and in some cases, to prevent pathology such as infection from spreading from one lung to another. Traditionally, this is accomplished by the placement of a double-lumen endotracheal tube device, with intermittent confirmation of proper position when necessary by use of a fiber optic bronchoscope.

In the placement of this device, a clinician initially guides the device blindly through the vocal cords after induction of general anesthesia, rotates it ninety degrees, and then advances it a few centimeters according to the tactile experience of the clinician. Once the clinician feels the device is in the correct position, a proximal, endotracheal balloon and a distal, endobronchial balloon are inflated. To confirm proper placement, the scope is then passed through the larger tracheal lumen to view the distal, endobronchial balloon, hopefully, to find the device properly positioned in the proximal portion of the main-stem bronchus of the lung to be ventilated.

During a surgery on one of the lungs, the patient is then positioned on his or her side, so that the surgical lung is in a nondependent position, accessible to the surgeon, and the contralateral, “healthy” lung is dependent. Because of the possibility of dislodgement during patient movement, the scope must be passed a second time to confirm proper tube position, and any necessary adjustments can be made. At any point during the operation, the tube can be accidentally withdrawn or advanced such that problems will arise with oxygenation, ventilation or deflation of the operative lung. This first requires detection by the clinician, and then the scope must be passed again to diagnose the problem and reposition the tube. Sometimes this is done under duress if the patient's condition is deteriorating. Early warning of the tube's malposition would make anesthetic management easier and improve patient safety.

Thus, there is a need for improved double lumen endotracheal tube devices and methods.

### SUMMARY OF THE DISCLOSURE

Embodiments described herein are directed toward systems, devices, and methods that provide visual input to a clinician during initial lung separation with the use of a double-lumen endotracheal tube device, and throughout an associated treatment procedure to facilitate the ongoing maintenance of a proper position of the double-lumen endotracheal tube. Visual input can be provided on a nearly continuous basis during insertion and/or throughout a procedure. Visual input can also be provided on demand without interrupting an ongoing procedure.

In accordance the present disclosure, various embodiments can comprise a double-lumen tubular device, e.g., a double-lumen endotracheal tube, and an access sheath positioned between the two lumens of the tube. The access sheath can be configured to slideably receive a medical device, such as an optical stylet device. The medical device can be connected to a video monitor, computer, other medical device, or the like. An optical stylet device connected to a video monitor, for example, can permit a clinician to continuously “see” the region of interest during the phases of insertion, positioning, and surgical manipulation.

Various other embodiments can comprise a double-lumen endotracheal tube and an optical system integrated into the walls of the tube, wherein the optical system can be connected to a video monitor so that a clinician continuously “sees” the region of interest, e.g. the carina, during the phases of insertion, positioning and surgical manipulation.

In accordance with another aspect of the disclosure, various embodiments comprise methods of use. For example, a method of use can comprise inserting a double-lumen endotracheal tube into a trachea and a mainstem bronchus and continuously providing a visual image of the region of interest on a monitor during insertion into the trachea and into the mainstem bronchus. Other methods can comprise continuously providing a visual image of the region of interest on a monitor during a thoracic surgical procedure.

### BRIEF DESCRIPTION OF THE DRAWINGS

The embodiments of the present disclosure will be described in conjunction with the accompanying drawing figures in which like numerals denote like elements and:

FIG. 1A illustrates a front view of an embodiment of a double-lumen endotracheal device;

FIG. 1B illustrates a transverse cross-sectional view of various embodiments of a double-lumen endotracheal device;

FIG. 1C illustrates a longitudinal cross-sectional view of a portion of a double-lumen endotracheal device comprising a retaining member;

FIG. 1D illustrates a longitudinal cross-sectional, perspective view of a portion of double-lumen endotracheal device comprising a retaining member;

FIGS. 2A illustrates a view from inside the mid-trachea, looking in a caudad direction at the carina and the two mainstem bronchi;

FIG. 3A illustrates a schematic view of an embodiment of an optical stylet device;

FIG. 3B illustrates a front view of an embodiment of a double-lumen endotracheal device having an access sheath;

FIG. 3C illustrates a magnified view of the distal end of the optical stylet embodiment depicted in FIG. 3A; and

FIG. 4 illustrates a front and schematic view of an embodiment of double-lumen endotracheal tube comprising an optical system.

### DETAILED DESCRIPTION

Persons skilled in the art will readily appreciate that various aspects of the present disclosure can be realized by any number of methods and apparatuses configured to perform the intended functions. Stated differently, other methods and apparatuses can be incorporated herein to perform the intended functions. It should also be noted that the accompanying drawing figures referred to herein are not all drawn to scale, but can be exaggerated to illustrate various aspects of the present disclosure, and in that regard, the drawing figures should not be construed as limiting. Finally, although the present disclosure can be described in connection with various principles and beliefs, the present disclosure should not be bound by theory.

Embodiments described herein are directed toward systems, devices, and methods that provide continuous visual input to a clinician during insertion of a medical device into the lumen or orifice of a body. In the instance of initial lung separation, continuous visual input can be provided during insertion of a double-lumen endotracheal tube device. Described embodiments can also provide continuous visual input throughout a treatment procedure to facilitate the ongoing maintenance of a proper position of a medical device, such as a double-lumen endotracheal tube.

In accordance with the present disclosure, various embodiments can comprise a dual-lumen tubular device, such as a double-lumen endotracheal tube, with an access sheath positioned between the two lumens. The access sheath can be configured to slideably receive a medical device, e.g., an optical stylet device further described herein. The medical device can be connected to a video monitor, computer, other medical device, or the like. An optical stylet connected to a video monitor, for example, can permit a clinician to continuously “see” the region of interest, e.g., the trachea, carina, bronchi, or the device placement or position during the phases of insertion, positioning, and surgical manipulation.

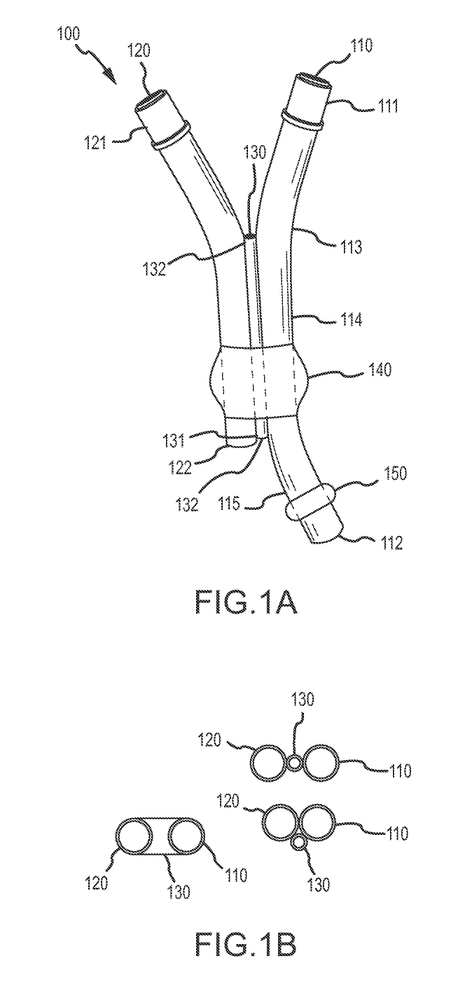

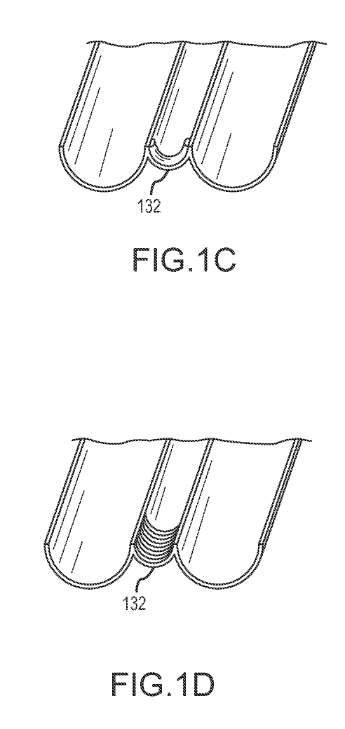

With reference to FIG. 1A, in accordance with various embodiments, double-lumen tubular device 100 can comprise a first tubular member 110; a second tubular member 120, wherein first tubular member 110 is connected to second tubular member 120 along at least a portion of their lengths; and an access sheath 130 longitudinally extending between first tubular member 110 and second tubular member 120. Access sheath 130 can be configured to receive medical device, such as an optical stylet device. Double-lumen tubular device 100 can further comprise an expandable and collapsible tracheal cuff 140 encompassing first tubular member, second tubular member, and access sheath 130, and/or a bronchial cuff 150 locatable along a distal portion of first tubular member 110. In an embodiment, double-lumen tubular device 100 is a double-lumen endotracheal tube comprising an access sheath.

First tubular member 110 and second tubular member 120 comprise any elongated structure of any shape having a proximal end 111, 121 and distal end 112, 122 (respectively) with a lumen there through. (“Distal” indicates the end of the tubular member that would be first inserted into the patient, and “proximal” indicates the end opposite therefrom.) First tubular member 110 can have a length greater than second tubular member 120. First tubular member 110 comprises a length sufficient to extend from within a mainstem bronchus out through a patient's nose or mouth. Second tubular member 120 comprises a length sufficient to extend from within a trachea out through a patient's nose or mouth.

Access sheath 130, first tubular member 110, and second tubular member 120 are integrated, coupled, connected, and/or fused (any of the foregoing referred to herein as “bundled”) to each other along a longitudinal direction and at least spanning a portion of access sheath 130 length. Cross-sectional views about this bundled region of various embodiments are illustrated in FIG. 1B. Access sheath 130, first tubular member 110, and second tubular member 120 can be bundled together by any connecting or bonding techniques known to the skilled artisan. Examples include, but are not limited to, solvent bonding, thermal adhesive bonding, and heat shrinking or sealing. Alternatively, access sheath 130, first tubular member 110, and second tubular member 120 can be integrated into a single manufactured piece that can be manufactured by techniques known to skilled artisan such as using known extrusion, injection molding, and other molding techniques.

During use, the proximal portions 113,123 of first and second tubular members 110, 120 can be locatable outside of the patient. These proximal portions can be adapted for a particular purpose, such as for connecting to a ventilation machine. Second tubular member 120 and first tubular member 110 need not be connected along this proximal portion 113, 123. The remaining portion of the second tubular member 120 can be bundled along an intermediate portion 114 of first tubular member 110. A distal portion of first tubular member 110 extends beyond the distal end 122 of second tubular member 120 and is referred to herein as an extending leg 115.

Distal end 131 of access sheath 130 terminates at a location which, upon use, would provide a view of a region of interest during the phases of insertion, positioning, and surgical manipulation. In the instance where the region of interest is the carina or mainstem bronchus after device 100 placement, access sheath is directed downward and distal end 131 of access sheath 130 can terminate near the distal end 122 of second tubular member 120, or extend beyond second tubular member 120. The distal end 131 of access sheath 130 can be bundled alongside first tubular member 110 or separate therefrom. Bundling with first tubular member 110 and extending beyond second tubular member 120 permits for the optical stylet device to have a view directly down a mainstem branch if desired. Unbundling from first tubular member 110 and extending beyond second tubular member 120 permits optical stylet device to have a freedom of motion. For example, a steerable optical stylet device can be utilized to provide various, adjustable views of the region(s) of interest, such as the bronchial balloon, trachea, carina, and mainstem branches. Distal end 131 of access sheath 130 can coterminate with second tubular member 120 or terminate thereabout, which permits a more aerial view of the carina along with at least a partial view of both mainstem bronchi.

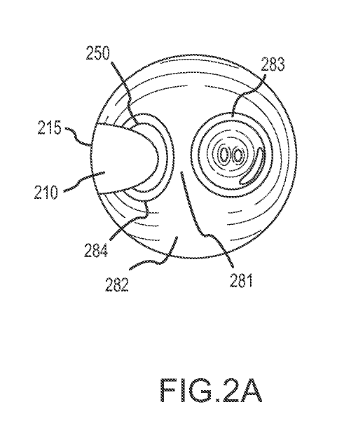

By way of example, FIG. 2A illustrates a view of an image which can be provided with use of various embodiments described herein. As illustrated, the carina 281, the posterior wall of the trachea 282, and the right bronchus 283 can be viewed along with extending leg 115 of first tubular member 110 positioned in the left bronchus 284.

Referring back to FIG. 1A, in an embodiment, extending leg 115 is configured to extend into a mainstem bronchus. Bronchial cuff 150 can be located within extending leg 115 and used to isolate a lung so that the lung can be selectively ventilated. As stated above, extending leg 115 can extend beyond second tubular member 120, and can also extend beyond the distal end of access sheath 130. Extending leg 115 can be slightly angled or configured to bend at a slight angle at its distal region to accommodate the geometry of the trachea relative to a mainstem bronchus.

The length of extending leg 115 is any length sufficient to permit positioning first tubular member 110 a certain depth within a mainstem bronchus. The specific length depends upon the size and anatomy of the patient. Typically, the length of the bronchi, which will contain at least a portion of the extending leg 115, ranges approximately 4-6 cm on the left and 1-2 cm on the right. The distance from bronchial cuff 150 to the opening of the tracheal lumen will be sufficient to permit ventilation of the contralateral lung.

Access sheath 130 comprises any receptacle configured to receive a medical device. When access sheath 130 is used for visualization purposes, access sheath 130 can be configured to receive an optical device and/or optical stylet device. Access sheath 130 can be positioned relative to the first tubular member 110 and second tubular member 120 so that when the optical device is inserted into access sheath 130, a clinician has a top view of the carina and at least a portion of the mainstem bronchus into which first tubular member 110 is being inserted, and more preferably both mainstem bronchi. For example, access sheath 130 can longitudinally extend between first tubular member 110 and second tubular member 120 and terminates about the same area as second tubular member 120.

Distal end 131 can permit light to pass through in both directions. Distal end 131 of access sheath 130 can comprise a retaining member 132 to ensure that a medical device does not extend beyond the distal end 131 of access sheath 130. Retaining member 132 can be configured not to obstruct light emitted from and received by a medical device. For example, retaining member can be a translucent, or preferably a transparent cap at the end 131 of access sheath 130. Retaining member 132 can be configured to be releasable so that the optical stylet device can be inserted beyond the distal end in order to obtain a different vantage point if desired. For example, as illustrated in FIG. 1C, retaining member 132 can comprise a raised surface projection that is tactilely detected and easily bypassed with a slight increase in insertion force.

Retaining member 132 can also comprise a locking mechanism which restricts movement in both directions. For example, as illustrated in FIG. 1D, retaining member 132 can comprise a threaded section. A medical device, such as an optical stylet device, can also comprise a threaded section so as to fix and/or permit finer control of its position when inserted and threaded into access sheath 130. Thus, by unthreading or further threading the vantage point can be adjusted.

Access sheath 130, first tubular member 110, and second tubular member 120 can comprise any bendable and elastic material, such as a soft polymer plastic and can comprise the same or different material. In an embodiment, the bendable and elastic material is suitable for its medical purpose, such as a medical grade or biocompatible material. For example, access sheath 130, first tubular member 110, and second tubular member 120 can comprise polyvinylchloride or a similar polymer. Any material used in the manufacture of endotracheal tube and double-lumen endotracheal tubes now known by a person of ordinary skill or hereafter provided can be used in the construction of device 100.

Both the first tubular membrane 110 and second tubular membrane 120 can comprise a connector at the proximal end 111, 121 configured to connect to a medical device, such as either a breathing system, e.g., an anesthesia machine, or to a CPAP (continuous positive airway pressure) apparatus, should the clinician desire to keep the surgical lung continuously inflated yet motionless, to improve the patient's oxygenation. For example, first tubular member 110 and second tubular member 120 can be coupled with a connector, such as a Y connector at the proximal end 111, 121.

In an embodiment, a double-lumen tubular device 100 can further comprise a bronchial cuff 150. Bronchial cuff 150 comprises an expandable and collapsible structure which encompasses the first tubular member 110 along the extending leg 115 to occlude a main stem when in an expanded state. For example, bronchial cuff 150 can comprise an annular balloon. In order to inflate bronchial cuff 150, device 100 can further comprise a first inflation lumen (not shown) that extends alongside bundle or is embedded within a first tubular member 110 or second tubular member 120 wall and is in fluid communication with bronchial cuff 150.

Similar to bronchial cuff 150, device 100 can also comprise a tracheal cuff 140. Tracheal cuff 140 comprises an expandable and collapsible structure which encompasses the bundled first tubular member 110, second tubular member 120, and access sheath 130 to occlude the trachea when in an expanded state. For example, tracheal cuff 140 can comprise an annular balloon. In order to inflate tracheal cuff 140, device 100 can further comprise a second inflation lumen (not shown) that extends alongside and is bundled or is embedded within a first tubular member 110 or second tubular member 120 wall and in fluid communication with tracheal cuff 140. First and second inflation lumens can be in fluid communication with each other to inflate cuffs 140, 150, or can be independent of each other.

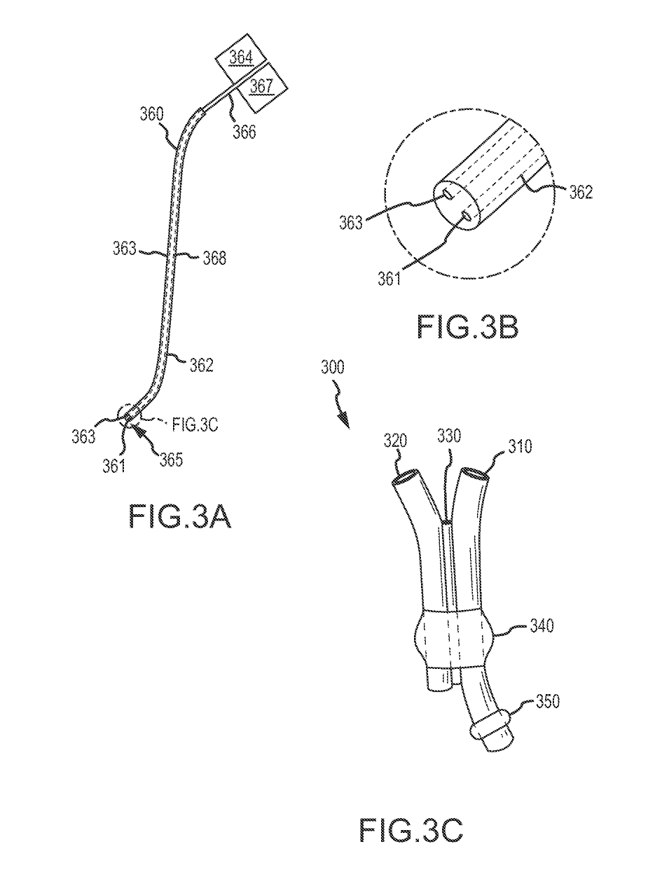

In accordance with the present disclosure, with reference to FIGS. 3A to 3C, various embodiments can also comprise double-lumen tubular device 300 comprising an access sheath 330 as described above and a releasable optical stylet device 360 configured to slide into access sheath 330 to provide an image of a region of interest throughout the phases of insertion, positioning, and surgical manipulation. Optical stylet device 360 comprises proximal 366 and distal end 365 with a viewing device 361 (e.g., an objective lens system) at the distal end 365 and an image guide 362 (e.g., an optical fiber) in optical communication with viewing device 361 and extending longitudinally within or about a bendable stylet 368. Image guide 362 is configured to optically connect to a video monitor 367 or an eyepiece lens (not shown). Video monitor 367 can be a small monitor mounted nearby on an IV pole so that the clinician can position for ease of use.

Optical stylet device 360 can also comprise an illumination channel 363 extending within or about bendable stylet 368 and optically coupled to an illumination source 364 to permit visible light, or another form of electromagnetic radiation, to be projected out distal end 365 of stylet 368.

U.S. Pat. No. 6,929,600 to Hill, entitled “Apparatus for Intubation,” is hereby incorporated by reference in its entirety. The video scope in the above-identified reference can be modified to a size which can be slideably received by access sheath 330.

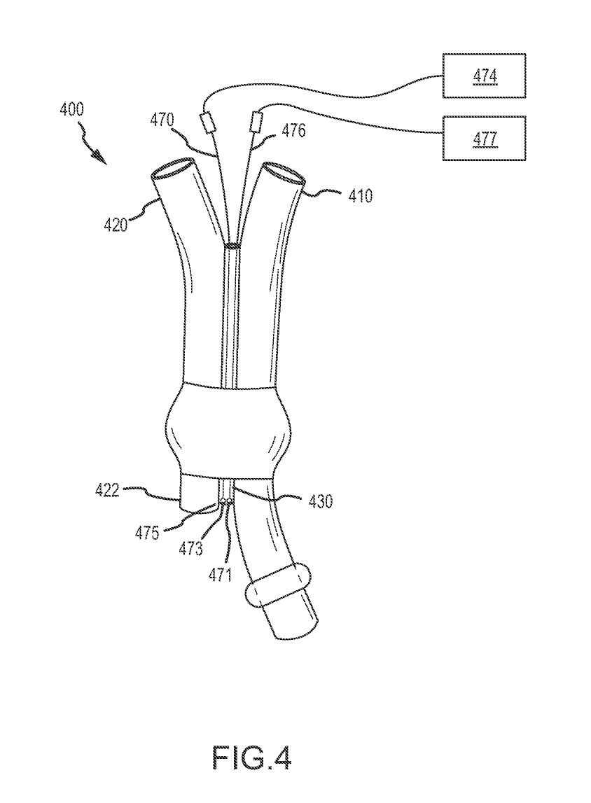

In lieu of access sheath 330 and a removable optical stylet device 360, an optical system can be integrated into the wall of a double-lumen endotracheal tube, preferably between a first tubular member and a second tubular member as described above. For example, with reference to FIG. 4, optical system 470 comprises proximal end 476 and distal end 475 with a viewing device 471 (e.g., an objective lens system) located at a distal end 475 near the distal end 422 of second tubular member 420 and an image guide 472 (e.g., an optical fiber) extending longitudinally within or along wall of double-lumen endotracheal tube 400 and in optical communication with viewing device 471. Image guide 472 is configured to optically connect to a video monitor 477 or an eyepiece lens (not shown). The video monitor 477 can be small monitor mounted nearby such as on an IV pole so that the clinician can position for ease of use.

Optical system 470 can also comprise an illumination channel 473 extending within or about wall of a first or second tubular member 410, 420 to terminate near the distal end 422 of second tubular member 420 and optically coupled to an illumination source 474 to permit visible light, or another form of electromagnetic radiation, to be projected in a caudad direction during use.

In accordance with the present disclosure, a method of use can comprise inserting into a trachea and a mainstem bronchus a double-lumen endotracheal tube and continuously providing a visual image of the region of interest, e.g. carina, on a monitor during insertion into trachea and into mainstem bronchus. Other methods of use can comprise continuously providing a visual image of the region of interest on a monitor during a surgical procedure. Surgical procedure can comprise a thoracic procedure on an isolated lung. The double-lumen endotracheal tube inserted into the trachea and mainstem bronchus can comprise an optical stylet device housed in an access sheath or an integrated optical system as described herein.

In accordance with the present disclosure, an exemplary method can comprise inserting an optical stylet device into an access sheath and connecting optical stylet device to a video monitor. After induction of general anesthesia, the double-lumen endotracheal tube and the optical stylet device can be inserted past the vocal cords. The optical stylet device and the access sheath are shaped and fitted such that the video image of the region of interest can be oriented correctly once the distal tip of the double-lumen endotracheal tube has passed into the mid-trachea and has been rotated ninety degrees counterclockwise. In this orientation, the tip of the first tubular member is poised above the left mainstem bronchus, and needs only to be advanced a few centimeters so that the bronchial cuff is visible on the monitor lying just beyond the carina. In a procedure wherein the right mainstem bronchus is to be occluded, this orientation would be reversed. At this point, both the tracheal cuff and the bronchial cuff are inflated with a small volume of air, in standard fashion. A breathing circuit is connected to the first tubular member and a second tubular member, and the double-lumen endotracheal tube is secured in place.

When the patient is turned into the lateral decubitus position, the clinician can easily check the video monitor to confirm that the second tubular member has not moved relative to the carina. He would then, as usual, open a port of the Y-connector attached to the nondependent lung to deflate it, simultaneously assuring that there was no gas leak coming from the ventilated dependent lung. The patient is then ready to be prepped and draped, with the carina on monitor in plain view throughout the surgery.

While an optical stylet device is inserted into the access sheath, the optical stylet device can be adjusted to adjust the vantage point. For example, the stylet can be adjusted by retracting or further advancing the stylet into the trachea. Additionally, the stylet can be steered such that the angle of the distal end is changed. The stylet can then be rotated as desired.

The present disclosure contemplates other medical devices configured for insertion into the lumen or orifice of a body comprising an access sheath. The access sheath being configured to receive an optical stylet as described herein to facilitate receiving visual input regarding an object of interest internally located.

The foregoing disclosure is merely illustrative of the present disclosure and is not intended to be construed as limiting the disclosure. Although one or more embodiments of the present disclosure have been described, persons skilled in the art will readily appreciate that numerous modifications could be made without departing from the spirit and scope of the present disclosure. As such, it should be understood that all such modifications are intended to be included within the scope of the present disclosure.

We claim:

1. A tubular device comprising
a first tubular member having a proximal and distal end, and a first lumen there through,
a second tubular member having a proximal and distal end, and a second lumen there through; and
an access sheath having a proximal and distal end and longitudinally extending between the first lumen and the second lumen;
wherein the distal end of first tubular member extends beyond the distal end of the second tubular member.

2. The tubular device of claim 1, wherein the distal end of the access sheath terminates at or near the distal end of the second tubular member.

3. The tubular device of claim 2 further comprising an inflatable and deflatable tracheal cuff.

4. The tubular device of claim 3 further comprising an inflatable and deflatable bronchial cuff.

5. The tubular device of claim 4 further comprising an inflation lumen to inflate the tracheal cuff and an inflation lumen to inflate the bronchial cuff.

6. The tubular device of claim 2, further comprising an optical stylet, wherein the optical stylet comprises an image device, an image guide, an illumination channel wherein the optical image guide is connectable to a monitor and the illumination channel is connectable to an illumination source and wherein the optical stylet is insertable into the access sheath.

7. The tubular device of claim 2, wherein the access sheath comprises a retaining member.

8. A method of using a double-lumen endotracheal tube comprising the steps of
inserting a double-lumen endotracheal tube into a trachea and a mainstem bronchus and
continuously providing a visual image of a region of interest on a monitor during insertion into the trachea and into the mainstem bronchus.

9. The method of claim 8 further comprising the step of continuously providing a visual image of the region of interest on a monitor during a surgical procedure.

10. The method of claim 9, wherein the surgical procedure comprises a thoracic procedure on an isolated lung.

11. The method of claim 8, wherein the double-lumen endotracheal tube comprises an access sheath and an optical stylet slideably housed therein.

12. The method of claim 8, wherein the double-lumen endotracheal tube comprises an optical system embedded therein.

13. The method of claim 8, wherein the region of interest is a carina.

14. A double-lumen endotracheal tube comprising
a first tubular member having a proximal and distal end, and a first lumen there through;
a second tubular member having a proximal and distal end, and a second lumen there through; and
an optical system having a proximal and distal end and longitudinally extending between the first lumen and the second lumen;
wherein the distal end of first tubular member extends beyond the distal end of the second tubular member.

15. The endotracheal tube of claim 14, wherein the distal end of the optical system terminates at or near the distal end of the second tubular member.

16. The endotracheal tube of claims 14, wherein the optical system comprises an image device, an image guide, an illumination channel wherein the optical image guide is connectable to a monitor and the illumination channel is connectable to an illumination source.

17. The endotracheal tube of claim 14 further comprising an inflatable and deflatable tracheal cuff.

18. The endotracheal tube of claim 16 further comprising an inflation lumen to inflate the tracheal cuff.

19. The endotracheal tube of claim 17 further comprising an inflatable and deflatable bronchial cuff.

20. The endotracheal tube of claim 18 further comprising an inflation lumen to inflate the bronchial cuff.
